(19) United States
(12) Patent Application Publication
(10) Pub. No.: US 2026/0363974 A1
(43) Pub. Date: Nov. 19, 2026

(54) NANO-HYDROXYAPATITE PROPHYLAXIS PASTE FOR DENTAL APPLICATION

(71) Applicant: Dr. Jen Natural, LLC, Mendota, MN (US)

(72) Inventor: Jennifer Eisenhuth, Mendota Heights, MN (US)

(21) Appl. No.: 19/679,480

(22) Filed: May 15, 2026

Related U.S. Application Data
(60) Provisional application No. 63/806,580, filed on May 15, 2025.

(51) Int. Cl. A61K 8/24; A61K 8/19; A61K 8/25; A61Q 11/00

(52) U.S. Cl. CPC A61K 8/24; A61K 8/19; A61K 8/25; A61Q 11/00; A61K 2800/28; A61K 2800/413; A61K 2800/48

(57) ABSTRACT

An oral care composition for polishing and conditioning tooth enamel includes nano‑hydroxyapatite particles, at least one abrasive agent, and a carrier including water and at least one humectant. The composition has a relative dentin abrasivity value of less than about 120 and deposits mineral material on the enamel surface during polishing. The nano‑hydroxyapatite may be derived from a natural calcium source such as eggshell material. A method of polishing tooth enamel includes applying the composition and mechanically polishing, during which nano‑hydroxyapatite fills surface micro‑defects, reduces roughness, and inhibits subsequent plaque or stain attachment. A polishing paste embodiment has an RDA value from about 40 to about 100.

### RELATED APPLICATIONS

This application claims the benefit of U.S. Provisional Patent Application No. 63/806,580, filed May 15, 2025, titled “NANO-HYDROXYAPATITE PROPHYLAXIS PASTE FOR DENTAL APPLICATION,” the entirety of the disclosure of which is hereby incorporated by this reference.

### TECHNICAL FIELD

This document relates to generally to oral care compositions and, more particularly, to enamel‑polishing compositions including nano‑hydroxyapatite and abrasive agents for cleaning, polishing, and conditioning tooth enamel while mitigating enamel loss.

### BACKGROUND

Maintaining the health and appearance of tooth enamel is an important aspect of oral care. Enamel surfaces are routinely subjected to mechanical cleaning and polishing processes intended to remove plaque, stains, and surface discoloration. These processes may employ abrasive compositions that mechanically alter the enamel surface to achieve a polished appearance.

While abrasive action can be effective for cleaning and polishing, mechanical interaction with enamel can also produce unintended effects at the microscopic level. Polishing operations may generate micro‑scratches, surface roughness, or localized removal of enamel material, particularly when repeated over time. Such surface changes can contribute to increased enamel wear, post‑polishing sensitivity, and enhanced susceptibility to re‑attachment of plaque or stains.

Compositions used for enamel polishing may therefore face competing technical considerations. On the one hand, sufficient abrasivity is desired to achieve effective cleaning and polishing. On the other hand, excessive or poorly controlled abrasivity can negatively affect enamel integrity. Achieving an appropriate balance between cleaning performance and enamel preservation presents a continuing technical challenge. In addition, enamel surfaces naturally contain micro‑defects, pores, and irregularities that may be exposed or exacerbated during mechanical polishing.

### SUMMARY

In some embodiments, the present disclosure is directed to oral care compositions for polishing and conditioning tooth enamel. The compositions described herein may include nano‑hydroxyapatite particles together with one or more abrasive agents dispersed in a carrier system. The carrier system may include water and at least one humectant, and may further include one or more thickening agents to provide a paste‑like consistency suitable for application to tooth surfaces.

In some embodiments, the nano‑hydroxyapatite particles may have particle sizes selected to interact with enamel micro‑structures. For example, the nano‑hydroxyapatite particles may have an average particle size on the order of tens of nanometers, such that the particles are capable of depositing mineral material into surface micro‑defects of the enamel during polishing. The nano‑hydroxyapatite may be present in an amount sufficient to influence enamel surface characteristics while the abrasive agents perform a polishing function.

The abrasive agents included in the compositions may include, in some embodiments, one or more of pumice, calcium carbonate, hydrated silica, or similar particulate materials, alone or in combination. The abrasive agents may be selected and formulated to provide polishing action while maintaining controlled abrasivity. In some embodiments, the compositions may have a relative dentin abrasivity (RDA) value below a selected threshold, such as below about 120. In certain embodiments, polishing pastes according to the disclosure may have RDA values in a range from about 40 to about 100.

In some embodiments, the compositions may be configured such that the presence of nano‑hydroxyapatite reduces net enamel material removal during polishing relative to compositions that lack nano‑hydroxyapatite. Rather than relying solely on mechanical removal of material, the compositions described herein may polish enamel while depositing mineral material onto the enamel surface, thereby addressing micro‑scratches, pores, or irregularities created or exposed during polishing.

The disclosure further includes, in some embodiments, methods of polishing tooth enamel. Such methods may include applying an oral care composition containing nano‑hydroxyapatite and at least one abrasive agent to a tooth surface, followed by mechanical polishing. During polishing, nano‑hydroxyapatite particles may deposit mineral material into surface micro‑defects of the enamel. The polishing may result in a reduction in enamel surface roughness, filling of micro‑scratches or micro‑fissures, reduced enamel sensitivity following polishing, and/or reduced subsequent attachment of plaque or stains.

In some embodiments, the oral care compositions may include additional components such as non‑cariogenic sweeteners, flavoring agents, preservatives, or stabilizers. For example, a non‑cariogenic sweetener such as xylitol may be included to improve sensory characteristics without contributing to tooth decay. Humectants such as glycerin or sorbitol may be included to maintain moisture and stability of the composition, and thickening agents such as cellulose derivatives may be used to achieve desired rheological properties.

In some embodiments, the nano‑hydroxyapatite may be derived from a natural calcium source. The natural calcium source may include eggshell material, which may provide hydroxyapatite along with accompanying trace minerals. The hydroxyapatite may be processed and reduced to nano‑scale particle sizes suitable for inclusion in the oral care compositions described herein.

Through the combination of controlled abrasivity and mineral deposition, the compositions and methods described in this disclosure may provide effective enamel polishing while mitigating enamel loss and conditioning the enamel surface during and after polishing.

The foregoing and other aspects, features, and advantages will be apparent from the DESCRIPTION and CLAIMS.

### DETAILED DESCRIPTION

The following detailed description provides numerous specific details. Those skilled in the relevant arts understand that embodiments of the disclosure may be practiced without these specific details. The disclosure may also be practiced in different and alternative configurations.

Unless specifically noted, it is intended that the words and phrases in the specification and the claims be given their plain, ordinary, and accustomed meaning to those of ordinary skill in the applicable arts. The singular forms “a,” “an,” and “the” include plural referents unless the context clearly dictates otherwise. Thus, for example, a reference to “a step” includes a reference to one or more of such steps. The words “exemplary,” “example,” “embodiment,” or various forms thereof are used herein to mean serving as an example, instance, or illustration. Any aspect or feature described herein as “exemplary” or as an “example” is not necessarily to be construed as preferred or advantageous over other aspects or designs. The examples are provided solely for purposes of clarity and understanding and do not limit or restrict the disclosure. It is to be appreciated that a myriad of additional or alternate examples of varying scope could have been presented, but have been omitted for purposes of brevity.

Throughout the description and claims of this specification, the words “comprise” and “contain” and variations of the words, for example “comprising” and “comprises”, mean “including but not limited to”, and are not intended to (and do not) exclude other components.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one.

Embodiments of the present disclosure are directed to oral care compositions that combine nano‑hydroxyapatite particles with one or more abrasive agents in a carrier system to polish and condition tooth enamel. Unlike conventional prophylaxis pastes that rely primarily on mechanical abrasion, the compositions described herein simultaneously polish enamel and deposit mineral material onto the enamel surface. The compositions therefore address surface micro‑defects — such as micro‑scratches, micro‑fissures, pores, and other irregularities — that are created, exposed, or exacerbated during mechanical polishing. The compositions are provided as pastes suitable for professional dental use, although the disclosure is not limited to any particular clinical or consumer context.

Hydroxyapatite is a naturally occurring mineral form of calcium apatite and a principal constituent of human tooth enamel. Nano‑hydroxyapatite (nHAp) refers to hydroxyapatite particles having dimensions on the nanometer scale. Because of its structural similarity to natural enamel, nano‑hydroxyapatite exhibits biocompatibility with tooth surfaces and integrates with enamel microstructures when applied under mechanical action.

The nano‑hydroxyapatite particles have an average particle size in a range of about 10 nm to about 200 nm. Narrower ranges are also contemplated, including about 20 nm to about 100 nm, about 20 nm to about 60 nm, about 30 nm to about 80 nm, and about 40 nm to about 100 nm. Particle sizes in these ranges may be selected to facilitate interaction with enamel micro‑structures, including penetration into surface pores, micro‑scratches, and other micro‑defects present on or near the enamel surface.

The nano‑hydroxyapatite particles may be present at about 5% to about 20% by weight of the composition. A concentration of about 10% by weight may be suitable for many applications. Other contemplated ranges include about 5% to about 10%, about 8% to about 15%, and about 12% to about 20% by weight. The concentration may be selected to provide sufficient mineral deposition during polishing while maintaining desirable paste consistency and handling characteristics.

During polishing, nano‑hydroxyapatite particles deposit mineral material into surface micro‑defects of the enamel. This mineral deposition fills micro‑scratches, micro‑fissures, pores, and other irregularities, resulting in a smoother enamel surface following polishing. A smoother post‑polishing surface may, in turn, reduce the tendency for plaque, stains, and other deposits to attach or re‑attach to the enamel. The presence of nano‑hydroxyapatite also reduces net enamel material removal during polishing relative to an otherwise identical composition that lacks nano‑hydroxyapatite. Rather than relying solely on subtractive mechanical action, the compositions described herein achieves a net balance between material removed by the abrasive agents and material deposited by the nano‑hydroxyapatite.

The nano‑hydroxyapatite may be derived from a natural calcium source. One such source is eggshell material. Eggshells are a renewable and abundant resource that naturally contain calcium, phosphate, and trace minerals including magnesium, zinc, iron, silicon, and collagen, among others. Hydroxyapatite derived from eggshell material may retain one or more of these trace minerals following processing, which may contribute to biocompatibility and integration with natural enamel.

The eggshell material may be poultry eggshell material. The eggshells may be collected from renewable sources and processed to isolate hydroxyapatite. A non‑limiting processing approach may include sourcing eggshells, cleaning the eggshells, sterilizing the eggshells, processing the eggshells to isolate hydroxyapatite, and reducing the hydroxyapatite to nano‑scale particle sizes while preserving magnesium, zinc, and other minerals. The resulting nano‑hydroxyapatite particles may then be incorporated into the oral care compositions described herein.

Eggshell‑derived nano‑hydroxyapatite offer advantages relative to synthetically produced alternatives. The natural mineral composition of eggshell‑derived nHAp enhance bioavailability of calcium and phosphate ions. Naturally occurring magnesium may support calcium absorption into enamel, and zinc may contribute antibacterial properties that reduce plaque buildup and support gum health. The use of eggshell‑derived nHAp may also reduce the environmental impact associated with synthetic hydroxyapatite manufacturing by repurposing a byproduct of the food industry.

Eggshell‑derived nano‑hydroxyapatite may outperform synthetically produced nano‑hydroxyapatite in certain respects. Because eggshells naturally contain calcium, phosphate, magnesium, iron, silicon, zinc, and collagen, among other minerals, the hydroxyapatite isolated from eggshells may retain a naturally integrated mineral profile that is not replicated by synthetic manufacturing processes. This natural mineral composition may enhance the bioavailability of calcium and phosphate ions relative to synthetic alternatives, supporting more effective mineral deposition into enamel micro‑defects during polishing.

Magnesium naturally present in eggshell‑derived nano‑hydroxyapatite may strengthen enamel by enhancing calcium transfer and absorption into the enamel crystal lattice, promoting deeper and faster mineral integration during and after polishing. Zinc naturally present in eggshell‑derived nano‑hydroxyapatite may contribute antibacterial properties that reduce plaque buildup, fight bacteria, and support gum health. The combination of nano‑hydroxyapatite, magnesium, and zinc derived from a single eggshell source may therefore provide a synergistic blend of remineralizing, antibacterial, and enamel‑strengthening effects in the oral care compositions described herein.

Eggshell‑derived nano‑hydroxyapatite has demonstrated biocompatibility with human tissues. Additionally, eggshell‑derived nHAp may be naturally structured to integrate seamlessly with human enamel, mimicking its natural crystallographic arrangement for effective remineralization and reduction of oral sensitivity.

Studies have also reported that hydroxyapatite formulated from eggshell sources can promote the formation of tubular enamel structures, with regular tubular arrangements appearing in hydroxyapatite‑formulated compositions relative to fluoride‑based compositions. The use of eggshell‑derived nano‑hydroxyapatite may also align with environmentally conscious practices by repurposing a byproduct of the food industry that would otherwise be discarded as waste. This sourcing approach may minimize waste and reduce the environmental and carbon footprint associated with synthetic nano‑hydroxyapatite manufacturing, which typically requires resource‑intensive chemical synthesis processes.

The disclosure is not limited to eggshell‑derived nano‑hydroxyapatite. Synthetically produced nano‑hydroxyapatite, or nano‑hydroxyapatite derived from other natural calcium sources — such as bovine bone, coral, or other mineral deposits — may also be used.

According to some embodiments, the compositions include at least one abrasive agent to provide mechanical polishing action. The abrasive agent or agents remove plaque, stains, surface discoloration, and other deposits from the enamel surface. Suitable abrasive agents include pumice, calcium carbonate, hydrated silica, and combinations thereof. Pumice may include finely graded volcanic ash. Calcium carbonate may also be referred to as chalk. Other abrasive agents that may be used, alone or in combination, include alumina, perlite, dicalcium phosphate, sodium bicarbonate, and similar particulate materials known in the art for dental polishing.

The abrasive agent particles may have an average particle size from about 1 µm to about 25 µm. Narrower ranges are also contemplated, including about 1 µm to about 10 µm, about 5 µm to about 15 µm, and about 10 µm to about 25 µm. Finer particle sizes generally provide a gentler polish, while coarser particle sizes provide more aggressive cleaning. The compositions may accordingly be provided in fine, medium, or coarse grit levels depending on the intended clinical application.

The abrasive agents may be present at a concentration sufficient to achieve effective polishing while maintaining controlled abrasivity. Suitable concentrations include about 30% to about 60% by weight, although amounts of about 10% to about 50% or about 20% to about 40% by weight may also be used depending on the intended application.

The nano‑hydroxyapatite particles and abrasive agents are dispersed in a carrier. The carrier includes water and at least one humectant. The carrier provides a medium for uniform distribution of the solid components and contributes to the consistency, moisture retention, and stability of the composition. Water may be present at about 20% to about 40% by weight, although other levels suitable for achieving the desired consistency are contemplated.

The humectant may include glycerin, sorbitol, or both. Other suitable humectants include propylene glycol, polyethylene glycol, and other polyols known in the art. Humectants maintain moisture and prevent the composition from drying out during storage and use. They also facilitate application of the composition to tooth surfaces. The humectant or humectants may be present at about 10% to about 25% by weight.

The compositions may further include at least one thickening agent to provide a paste‑like consistency suitable for application to tooth surfaces. The thickening agent also contributes to stability during storage and use. Suitable thickening agents include cellulose derivatives such as hydroxyethyl cellulose, hydroxypropyl cellulose, hydroxypropyl methylcellulose, sodium carboxymethyl cellulose, and cellulose gum. Other thickening agents — including xanthan gum, carbomer, and similar rheology modifiers — may also be used. The thickening agent may provide sufficient viscosity such that the composition remains intact when transferred from a dispensing container or tray to a polishing implement.

The compositions may include one or more additional components to enhance functionality, stability, or sensory characteristics.

A non‑cariogenic sweetener — that is, a sweetener that does not contribute to tooth decay — may be included. Xylitol is one suitable non‑cariogenic sweetener. Xylitol may improve taste and patient acceptability and may also help reduce the growth of cavity‑causing bacteria and aid in saliva production. Other non‑cariogenic sweeteners, such as stevia, erythritol, and sorbitol (which may also function as a humectant), are also contemplated.

Flavoring agents may include natural or artificial flavors such as peppermint, spearmint, wintergreen, anise, or fruit flavors. Natural flavor may be present at less than about 1% by weight. Preservatives and stabilizers may be included as needed to maintain formulation integrity during storage. These may be selected from those known in the art for use in oral care products. The compositions may be fluoride‑free, which may be preferred by patients or practitioners seeking non‑fluoride alternatives for professional prophylaxis. Alternatively, fluoride may optionally be included.

The abrasivity of the compositions may be characterized by a relative dentin abrasivity (RDA) value. RDA is a standardized measure of the erosive effect of an abrasive composition on dentin, with lower RDA values generally corresponding to less abrasive compositions.

The compositions may have an RDA value of less than about 120. Compositions configured as polishing pastes may have RDA values from about 40 to about 100. Narrower ranges are also contemplated, including about 40 to about 70, about 50 to about 80, and about 60 to about 100. The RDA value may be influenced by the type, particle size, concentration, and hardness of the abrasive agents, as well as the presence of nano‑hydroxyapatite and other components. The compositions may be formulated to provide effective polishing and stain removal while maintaining abrasivity below a threshold associated with excessive enamel wear.

The oral care composition may be provided as a polishing paste that includes nano‑hydroxyapatite particles, at least one abrasive agent, and a paste carrier. The paste carrier may include water, one or more humectants, and one or more thickening agents, as described above. The polishing paste may have a viscosity and consistency suitable for application to tooth surfaces using a polishing implement.

The polishing paste may be configured for use with a rotary polishing implement, such as a rubber cup, a prophy cup, a bristle brush, or other rotary attachments used in dental prophylaxis procedures. The polishing paste may also be applied manually or using other types of polishing devices.

The polishing paste may have an RDA value from about 40 to about 100. The nano‑hydroxyapatite particles may be present at about 5% to about 20% by weight, and the abrasive agent may include particles having an average particle size from about 1 µm to about 25 µm.

The oral care compositions described herein may be prepared by combining the nano‑hydroxyapatite particles, abrasive agents, and carrier components using mixing techniques suitable for achieving uniform dispersion of solids within the carrier system.

In one approach, a carrier pre‑mixture may be prepared by combining water, humectant (such as glycerin and/or sorbitol), and thickening agent (such as a cellulose derivative) under mixing sufficient to dissolve or hydrate the thickening agent and form a homogeneous carrier base. Mixing may be performed at ambient temperature using conventional mixing equipment, such as a planetary mixer, overhead stirrer, or similar apparatus. The carrier pre‑mixture may be mixed until a uniform, lump‑free base of desired viscosity is achieved.

Abrasive agents (such as pumice, calcium carbonate, and/or hydrated silica) may then be incorporated into the carrier pre‑mixture under continued agitation. The abrasive agents may be added incrementally or as a single addition, with mixing continued until the abrasive particles are uniformly distributed throughout the carrier. In some approaches, the abrasive agents may be pre‑blended as a dry mixture before incorporation into the carrier.

Nano‑hydroxyapatite particles may be incorporated into the composition during or after incorporation of the abrasive agents. The nano‑hydroxyapatite may be added as a dry powder or as a pre‑dispersed slurry in a portion of the carrier system. Agitation sufficient to maintain dispersion of the nano‑hydroxyapatite particles may be employed, while avoiding excessive shear that could damage particle integrity or promote undesirable agglomeration. One or more non‑toxic stabilizing agents — such as lecithins, polysorbate‑type materials, or poloxamers — may be included at levels sufficient to maintain dispersion uniformity of the nano‑hydroxyapatite within the composition.

Optional additives, including non‑cariogenic sweeteners (such as xylitol), flavoring agents, preservatives, and stabilizers, may be introduced during or after the dispersion step. The order of addition of optional additives may be varied as appropriate without departing from the scope of the disclosure.

The resulting composition may be adjusted to a viscosity appropriate for paste‑like application to tooth surfaces. Brief deaeration — for example, by vacuum mixing or centrifugation — may be employed to reduce entrained air and improve paste uniformity. Filtration or screening may be performed to reduce aggregates and ensure consistent particle distribution.

The compositions may be provided in fine, medium, or coarse grit formulations by varying the particle size and concentration of the abrasive agents during preparation. The nano‑hydroxyapatite concentration may be adjusted independently of the abrasive agent concentration to achieve a desired balance between mineral deposition and polishing performance.

The order of addition may be varied without departing from the scope of the disclosure. For example, the nano‑hydroxyapatite may be incorporated before the abrasive agents, or the nano‑hydroxyapatite and abrasive agents may be pre‑blended as a dry mixture before incorporation into the carrier. The carrier components may also be added in a different sequence, such as adding the humectant to water before introducing the thickening agent, or adding the thickening agent directly to the humectant before diluting with water.

Where the nano‑hydroxyapatite is derived from eggshell material, the eggshell‑derived nano‑hydroxyapatite may be prepared according to the following non‑limiting approach. Eggshells — which may be poultry eggshells — may be collected from renewable sources. The collected eggshells may be cleaned to remove membrane material, debris, and organic residue. The cleaned eggshells may then be sterilized, such as by autoclaving, dry‑heat sterilization, chemical sterilization, or other suitable methods. The sterilized eggshells may be processed to isolate hydroxyapatite, such as by calcination, acid‑base reaction, hydrothermal processing, or mechanical and chemical extraction techniques. The isolated hydroxyapatite may then be reduced to nano‑scale particle sizes — for example, by ball milling, wet grinding, sonication, or other size‑reduction techniques — while preserving naturally occurring magnesium, zinc, and other essential trace minerals. The resulting eggshell‑derived nano‑hydroxyapatite particles may then be incorporated into the oral care compositions as described above.

The disclosed methods of preparation are not limited to any particular equipment, mixing speed, temperature, order of addition, or processing conditions. Variations and adjustments consistent with achieving uniform dispersion and desired paste characteristics are contemplated and within the scope of the disclosure.

The present disclosure also includes methods of polishing tooth enamel. A method may include applying to a tooth surface an oral care composition including nano‑hydroxyapatite particles and at least one abrasive agent, and mechanically polishing the tooth surface. During polishing, the nano‑hydroxyapatite may deposit mineral material into surface micro‑defects of the enamel. The composition may be applied using any suitable applicator or implement. A rotary polishing implement — such as a rubber cup or prophy cup driven by a slow‑speed handpiece — is one suitable option. The composition may also be applied using a bristle brush, a finger applicator, or other means. The method may be performed during a professional dental cleaning or prophylaxis procedure, or in a non‑professional or at‑home context.

Mechanical polishing may include rotation, oscillation, or other movement of the polishing implement against the tooth surface while the composition is present. During such mechanical action, the abrasive agents remove plaque, stains, tartar, and surface deposits from the enamel, while the nano‑hydroxyapatite particles deposit mineral material into micro‑defects of the enamel surface. The polishing may produce a reduction in enamel surface roughness relative to the pre‑polished enamel surface — that is, the enamel surface may be smoother after polishing than it was before. This reduction in roughness may result from the combined effects of mechanical polishing and mineral deposition by the nano‑hydroxyapatite.

The polishing may also fill micro‑scratches or micro‑fissures formed during mechanical polishing. Mechanical polishing inherently generates micro‑scale surface features, including micro‑scratches, on the enamel. The nano‑hydroxyapatite may deposit into these features as they are formed, filling them with mineral material and reducing their depth and prominence.

The polishing may result in reduced enamel sensitivity following completion. Without intending to be bound by theory, deposition of nano‑hydroxyapatite into surface micro‑defects may occlude pathways through which stimuli reach dentin, thereby reducing post‑polishing sensitivity. The compositions may accordingly be suitable for patients with dentin hypersensitivity, xerostomia, or high caries risk. The polishing may further reduce subsequent attachment of plaque or stains to the enamel surface. A smoother enamel surface, resulting from mineral deposition into micro‑defects, may present fewer sites for mechanical retention of plaque, bacteria, stains, and other deposits. This may extend the duration of the polishing effect and delay re‑accumulation of surface deposits.

The following exemplary formulations are provided for illustration and are not intended to limit the scope of the disclosure.

Example 1 — Fine‑Grit Prophylaxis Paste. A polishing paste including about 10% by weight nano‑hydroxyapatite (average particle size about 20–60 nm), about 35% by weight pumice (fine grade, average particle size about 1–10 µm), about 25% by weight water, about 15% by weight glycerin, about 5% by weight sorbitol, about 3% by weight cellulose gum, about 5% by weight xylitol, and less than about 1% natural flavor. The paste has an RDA value of about 40 to about 60.

Example 2 — Medium‑Grit Prophylaxis Paste. A polishing paste including about 15% by weight nano‑hydroxyapatite (average particle size about 20–100 nm), about 40% by weight of a combination of pumice and calcium carbonate (medium grade, average particle size about 5–15 µm), about 20% by weight water, about 12% by weight glycerin, about 3% by weight hydroxyethyl cellulose, about 7% by weight xylitol, and less than about 1% natural flavor and preservative. The paste has an RDA value of about 60 to about 80.

Example 3 — Eggshell‑Derived nHAp Prophylaxis Paste. A polishing paste including about 10% by weight eggshell‑derived nano‑hydroxyapatite (average particle size about 20–100 nm, retaining naturally occurring trace minerals including magnesium and zinc), about 45% by weight pumice and hydrated silica (medium grade), about 20% by weight water, about 12% by weight glycerin, about 4% by weight cellulose gum, about 7% by weight xylitol, and less than about 1% natural flavor. The paste has an RDA value of about 50 to about 70.

Example 4 — Natural Extract Prophylaxis Paste. A polishing paste including about 10% by weight eggshell‑derived nano‑hydroxyapatite (average particle size about 20–100 nm), about 30% by weight of a combination of hydrated silica and calcium carbonate (fine to medium grade), about 20% by weight water, about 12% by weight glycerin derived from coconut, about 5% by weight xylitol, about 3% by weight cellulose gum, about 1% by weight stevia rebaudiana leaf extract, and less than about 1% each of natural peppermint flavor, natural spearmint flavor, peppermint oil, spearmint oil, peppermint menthol crystal, citric acid as a natural preservative, anise extract, papaya extract, black cherry extract, and glycyrrhizin (Chinese licorice root) extract. The paste may be formulated to be foam‑free, making it suitable for patients with mouth ulcers, sensitive gums, or other conditions where foaming agents may cause discomfort. The paste has an RDA value of about 40 to about 60.

Example 5 — Sensitivity‑Focused Prophylaxis Paste. A polishing paste including about 15% by weight eggshell‑derived nano‑hydroxyapatite (average particle size about 20–60 nm, retaining naturally occurring trace minerals including magnesium and zinc), about 25% by weight fine‑grade pumice (average particle size about 1–5 µm), about 25% by weight water, about 15% by weight glycerin, about 5% by weight sorbitol, about 3% by weight cellulose gum, about 7% by weight xylitol, and less than about 1% natural flavor. This formulation emphasizes a higher nano‑hydroxyapatite loading and finer abrasive grade to maximize mineral deposition and minimize mechanical enamel removal, and may be particularly suitable for patients with dentin hypersensitivity, xerostomia, or high caries risk. The paste has an RDA value of about 40 to about 55. The paste may be formulated to be fluoride‑free and foam‑free.

Example 6 — Minimal‑Ingredient Prophylaxis Paste. A polishing paste including about 10% by weight nano‑hydroxyapatite, about 40% by weight pumice (medium grade), about 25% by weight water, about 15% by weight glycerin, and about 7% by weight xylitol, with the balance being one or more thickening agents. This formulation includes a reduced ingredient count and may be suitable for patients who prefer compositions with fewer additives or who have sensitivities to certain flavoring or coloring agents. The paste has an RDA value of about 50 to about 75.

The following prophetic examples illustrate comparative testing that may be performed to evaluate the compositions described herein. These examples are provided for purposes of illustration and are not based on actual experimental data.

These examples may be varied by routine formulation adjustments — including selection of abrasive type and grade, humectant ratios, thickening agent type and concentration, and nano‑hydroxyapatite source and loading — without departing from the scope of the disclosure.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

Many additional implementations are possible. Further implementations are within the CLAIMS.

In places where the description above refers to particular implementations, it should be readily apparent that a number of modifications may be made without departing from the spirit thereof and that these implementations may be applied to other implementations disclosed or undisclosed. The presently disclosed are, therefore, to be considered in all respects as illustrative and not restrictive.

What is claimed is:

1. An oral care composition comprising:
nano‑hydroxyapatite particles;
at least one abrasive agent; and
a carrier comprising water and at least one humectant,
wherein the composition has a relative dentin abrasivity (RDA) value of less than about 120 and is configured to polish tooth enamel while depositing mineral material on an enamel surface.

2. The oral care composition of claim 1, wherein the nano‑hydroxyapatite particles have an average particle size in a range of about 10 nm to about 200 nm.

3. The oral care composition of claim 1, wherein the nano‑hydroxyapatite particles have an average particle size in a range of about 20 nm to about 100 nm.

4. The oral care composition of claim 1, wherein the at least one abrasive agent comprises at least one of pumice, calcium carbonate, hydrated silica, or combinations thereof.

5. The oral care composition of claim 1, wherein the presence of the nano‑hydroxyapatite reduces net enamel material removal during polishing relative to an otherwise identical composition lacking nano‑hydroxyapatite.

6. The oral care composition of claim 1, further comprising at least one thickening agent selected from cellulose derivatives.

7. The oral care composition of claim 1, wherein the at least one humectant comprises at least one of glycerin or sorbitol.

8. The oral care composition of claim 1, further comprising a non‑cariogenic sweetener.

9. The oral care composition of claim 8, wherein the non‑cariogenic sweetener comprises xylitol.

10. The oral care composition of claim 1, wherein the nano‑hydroxyapatite is derived from a natural calcium source.

11. The oral care composition of claim 10, wherein the natural calcium source comprises eggshell material.

12. A method of polishing tooth enamel, comprising:
applying to a tooth surface an oral care composition comprising nano‑hydroxyapatite particles and at least one abrasive agent, and
mechanically polishing the tooth surface,
wherein nano‑hydroxyapatite deposits mineral material into surface micro‑defects of the enamel during polishing.

13. The method of claim 12, wherein the polishing produces a reduction in enamel surface roughness relative to a pre‑polished enamel surface.

14. The method of claim 12, wherein the polishing fills micro‑scratches or micro‑fissures formed during mechanical polishing.

15. The method of claim 12, wherein the polishing results in reduced enamel sensitivity following completion of the polishing.

16. The method of claim 12, wherein the polishing reduces subsequent attachment of plaque or stains to the enamel surface.

17. A polishing paste comprising:
nano‑hydroxyapatite particles;
at least one abrasive agent; and
a paste carrier, wherein the polishing paste has a relative dentin abrasivity (RDA) value from about 40 to about 100.

18. The polishing paste of claim 17, wherein the paste is configured for use with a rotary polishing implement.

19. The polishing paste of claim 17, wherein the at least one abrasive agent comprises particles having an average particle size from about 1 µm to about 25 µm.

20. The polishing paste of claim 17, wherein the nano‑hydroxyapatite particles are present in an amount from about 5% to about 20% by weight of the paste.
